(19) United States
(12) Patent Application Publication
(10) Pub. No.: US 2021/0182705 A1
(43) Pub. Date: Jun. 17, 2021

(54) MACHINE LEARNING BASED SKIN CONDITION RECOMMENDATION ENGINE

(71) Applicant: 7 Trinity Biotech Pte. Ltd., Singapore (SG)

(72) Inventor: Robert Alexandar Bates, Byron Bay (AU)

(21) Appl. No.: 16/936,329

(22) Filed: Jul. 22, 2020

Related U.S. Application Data
(60) Provisional application No. 62/948,662, filed on Dec. 16, 2019.

(51) Int. Cl. G06N 5/04; G06N 20/00; A61B 5/00; G06T 7/00

(52) U.S. Cl. CPC G06N 5/04; G06N 20/00; A61B 5/7267; A61B 5/0064; A61B 5/442; G06T 2207/30088; G06T 7/0012; G06T 2207/30201; G06T 2207/20081; G06T 2200/24; A61B 5/7435

(57) ABSTRACT

A skin condition recommendation engine identifies skin conditions of a user's face and recommends actions and/or products that increase a likelihood that the skin conditions will be remedied. The skin condition recommendation engine trains a machine learned model using a training set of information that includes images and identified skin conditions of training users' faces. The skin condition recommendation engine inputs images of the user's face into the machine learned model, which outputs identified skin conditions of the user. The skin condition recommendation engine accordingly identifies actions that, if performed by the user, would increase a likelihood of the skin conditions being remedied. The skin condition recommendation engine modifies an interface of a device of the user to show the identified actions.

### CROSS REFERENCE TO RELATED APPLICATIONS

This application claims the benefit of U.S. Provisional Application No. 62/948,662, filed Dec. 16, 2019, which is incorporated by reference in its entirety.

### TECHNICAL FIELD

This disclosure generally relates to the field of skin care, and specifically to a machine learning-based skin condition recommendation engine.

### BACKGROUND

A user may periodically consult a dermatologist for a skin condition. Seeking medical attention regularly, however, can be expensive and impractical. Conventional mobile beauty applications are often limited to virtual make up and styling sessions, and do not provide users with access and suggestions to skin health improvement regimens.

### SUMMARY

A method, system, and non-transitory computer-readable medium for training and applying a machine-learned model configured to provide recommendations for improving skin conditions are described herein. A training set of information is accessed comprising, for each of a plurality of training users, an image of the training user's face and an identification of skin conditions of the training user. A machine learned model is trained based on the accessed training set of information, and is applied to received images of a user's face. The machine learned model identifies one or more skin conditions of the user. Actions are identified that, if performed by the user, increase a likelihood that the skin conditions will be remedied. Finally, an interface displayed by a device of the user is modified to include the identified actions.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1 illustrates a system environment of a skin condition recommendation engine, in accordance with one or more embodiments.

FIG. 2 illustrates training and applying a machine-learned model configured to provide recommendations for improving skin conditions, in accordance with one or more embodiments.

FIG. 3 illustrates an example process for providing a user with recommendations for improving skin conditions, in accordance with one or more embodiments.

FIGS. 4A-C illustrate example user interfaces through which the user may interact with the skin condition recommendation engine, in accordance with one or more embodiments.

### DETAILED DESCRIPTION OF DRAWINGS

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Overview

A user may use beauty applications on a client device for virtual styling recommendations, tips, or appointments with beauty specialists. The beauty applications do not enable the user to track changes and/or trends in skin conditions. The method and system included herein describe a skin condition recommendation engine that uses machine learning techniques to analyze images of the user's face regularly. The skin condition recommendation engine thereby identifies changes in the user's skin conditions over time and recommends products and/or actions for the user that increase a likelihood of the user's skin conditions being remedied.

### System Environment

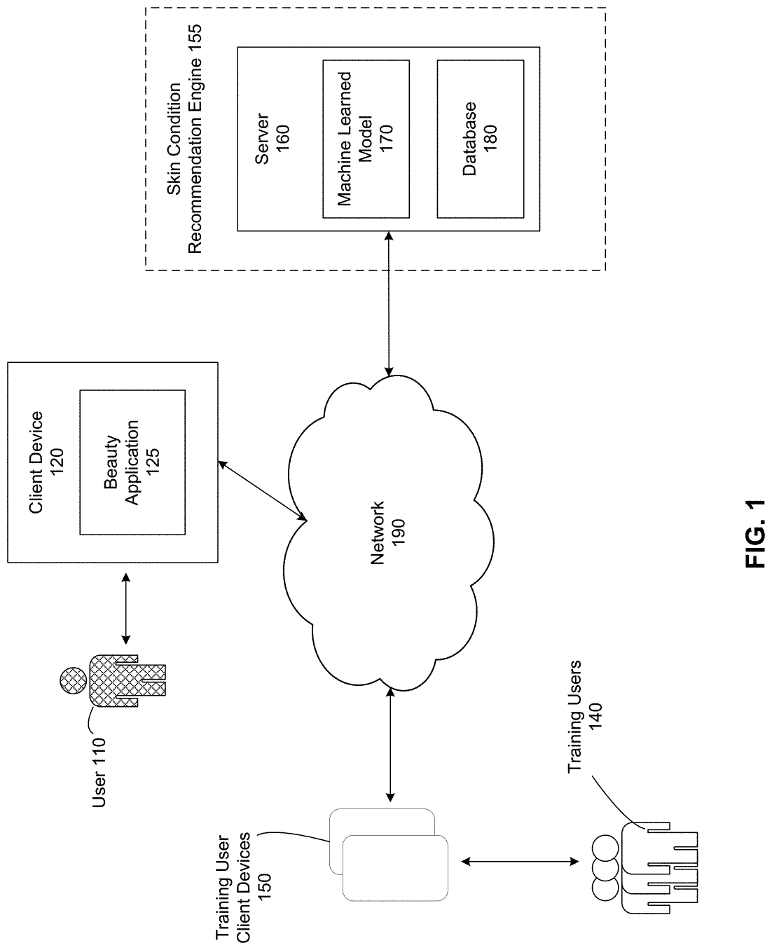

FIG. 1 illustrates a system environment of a skin condition recommendation engine, in accordance with one or more embodiments. The skin condition recommendation engine receives images of a user's face and provides the user with recommendations (such as product recommendations, action recommendations, and the like) that increase a likelihood that skin conditions of the user will improve. The system environment includes a user 110, a client device 120, a plurality of training users 140, a plurality of training user client devices 150, the skin condition recommendation engine 155, and a network 190.

The skin condition recommendation engine 155 provides recommendations to the user 110 to improve the likelihood that skin conditions of the user 110 will be remedied. The skin condition recommendation engine 155 takes, as input, a set of images of the face of the user 110, and from the set of images, one or more skin conditions associated with the user's face. The skin condition recommendation engine 155 outputs recommendations for the user 110 to help with improving the identified skin conditions. For example, recommendations may include product suggestions (e.g., topical lotions, ointments, dietary supplements), as well as action suggestions (e.g., washing the face, facials, etc.).

The client device 120 couples the user 110 to the skin condition recommendation engine 155. The client device 120 is a computing device capable of transmitting and/or receiving data over the network 190. The client device 120 may be a conventional computer (e.g., a laptop or a desktop computer), a cellphone, or a similar device that communicates with the skin condition recommendation engine 155. The client device 120 may be a device worn by the user 110 (e.g., a smart watch). In some embodiments, the client device 120 captures the set of images of the user's face via one or more cameras. The client device 120 may prompt the user 110 to take the images of the user's face and provide the images to the skin condition recommendation engine 155. In some embodiments, another device, such as an external camera, may couple to the client device 120 and provide the skin condition recommendation engine 155 with the images of the user's face. In some embodiments, multiple client devices 120 provide the skin condition recommendation engine 155 with the images of the user's face. The client device 120 presents the recommendations to the user 110 as well, via a user interface displayed on the client device 120. In some embodiments, the client device 120 may access one or more images of the user's face from an external data source (e.g., a social network profile of the user 110) and provide the images to the skin condition recommendation engine 155.

In some embodiments, the client device 120 includes and executes a beauty application 125. The beauty application 125 may host the skin condition recommendation engine 155. The beauty application 125 may include an artificially intelligent personal digital assistant and/or a social feed where a plurality of users of the beauty application 125 (e.g., including the user 110) can share images of their faces, recommendations, and earn social rewards for completing tasks, for example. In some embodiments, the beauty application 125 prompts the user 110 to periodically (e.g., once a day) capture “selfies,” which are images of the user's face taken via a front facing camera of the client device 120.

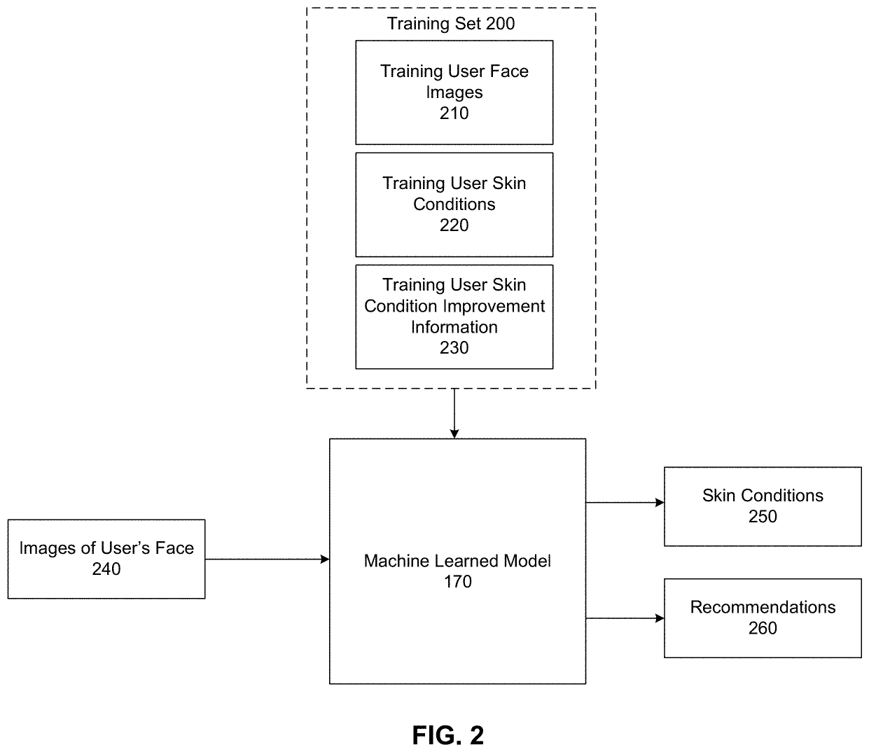

The skin condition recommendation engine 155 identifies skin conditions of and generates recommendations for the user 110 using the trained machine learned model 170. The machine learned model 170 is stored by the server 160 and trained using a training set of data including information about a plurality of training users 140. The training users 140 may be people other than the user 110 that use the skin condition recommendation engine 155. The training set includes, for each training user 140, images of faces of the training users 140, one or more known skin conditions associated with the faces of the training users 140, and products and actions that led to the improvement of the one or more skin conditions of the training users 140. The training and application of the machine learned model 170 is further described with respect to FIG. 2.

The training user client devices 150 provide the images of the training user's faces to the skin condition recommendation engine 155, over the network 190. The training user client devices 150 may be substantially similar to the client devices 120, and may be, for example, conventional computers or cellphones owned by each of the training users 140. In some embodiments, in response to capturing an image with a face of the training user 140, each client device 150 automatically adds the image to the training set. The training user client devices 150 may include the beauty application 125, through which the training users 140 can provide the images of their faces to the skin condition recommendation engine. In some embodiments, the beauty application 125 prompts the training users 140 to capture selfies which are added to a training set used to train the machine learned model 170.

The skin condition recommendation engine 155 includes the server 160. The server 160 stores and receives the set of images of the user's face from the client device 120 and the images of the training users' faces from the training user client devices 150. The server 160 hosts the machine learned model 170 and the database 180. The server 160 may be located on a local or remote physical computer and/or may be located within a cloud-based computing system.

The database 180 stores information relevant to the recommendation engine. The database 180 stores the images of the face of the user 110, the identified skin conditions of the user 110, and the training set comprising the images, skin conditions, and skin condition improvement information associated with the training users 140.

The network 190 transmits data from the client device 120 and the training user client devices 150 to the server 160 and vice versa. The network 190 may be a local area and/or wide area network using wireless and/or wired communication systems, such as the Internet. In some embodiments, the network 190 transmits data over a single connection (e.g., a data component of a cellular signal, or WiFi, among others) and/or over multiple connections. The network 190 may include encryption capabilities to ensure the security of consumer data. For example, encryption technologies may include secure sockets layer (SSL), transport layer security (TLS), virtual private networks (VPNs), Internet Protocol security (IPsec), etc.

### Training and Application of Machine Learned Model

FIG. 2 illustrates training and applying the machine-learned model 170, the machine learned model 170 configured to provide recommendations for improving skin conditions, in accordance with one or more embodiments. As described in FIG. 1, the machine learned model 170 takes in a set of images of a user's face (e.g., the face of the user 110), identifies one or more skin conditions associated with the user's face, and generates recommendations to improve the skin conditions.

The machine learned model 170 is trained using a set of training data (“training set 200”). The training set 200 includes training user face images 210 (e.g., images of faces of the training users 140), training user skin conditions 220 (e.g., one or more skin conditions associated with the faces of the training users 140), and training user skin condition improvement information 230 (e.g., information on how the training users 140 improved their one or more skin conditions). As described with respect to FIG. 1, the training user face images 210, training user skin conditions 220, and the training user skin condition improvement information 230 may be self-reported and/or captured by client devices (e.g., the training user client devices 150) coupled to the skin condition recommendation engine 155. In some embodiments, the skin condition recommendation engine 155 prompts and/or incentivizes training users to provide the training user face images 210, training user skin conditions 220, and the training user skin condition improvement information 230 by gamification, rewards (such as social network status awards), and/or product offers. For example, a training user may receive an offer on a product if they capture and provide an image of the training user's face every day for one month.

The training user face images 210 include a plurality of images of faces of training users (e.g., the training users 140). For a training user, the training user face images 210 includes images of the training user's face captured at regular intervals over a period of time (e.g., once in the morning and in the evening for one month, once every day for one month).

The training user skin conditions 220 include one or more skin conditions associated with the faces of the training users. The skin conditions include sensitive skin, oily skin, dry skin, combination (e.g., a combination of sensitive, oily, and/or dry skin) skin, and normal skin. In some embodiments, a combination skin condition includes a plurality of skin conditions may be associated with a training user's face (e.g., the training user's forehead may be oily, but the cheeks are dry). The combination skin condition may be represented by a coefficient (such as a coefficient between 0 and 1) representative of each component skin condition (e.g., a training user may have 0.7 dry skin and 0.3 oily skin). The skin conditions may be diagnosed by dermatologists, other doctors, self-reported by the training users, or some combination thereof.

The training user skin condition improvement information 230 includes information about products used by the training users and/or actions taken by the training users that improved their skin conditions. An improvement in skin conditions may be signified by a change in the skin condition to normal skin, and/or a reduction in the skin condition. For example, a training user may initially have dry skin, but may over time transition to normal skin, thereby signifying an improvement. In another example, a training user may go from fully oily skin (e.g., 1.0 oily skin) to 0.7 oily skin and 0.3 normal skin. In some embodiments, the level of improvement in skin conditions may be calculated by the skin condition recommendation engine 155. For example, a training user who goes from fully oily skin to 0.7 oily skin may have a skin improvement level of 0.3. In some embodiments, the skin condition recommendation engine requires a threshold level before rendering an improvement in skin conditions. The training user skin condition improvement information 230 also includes a timeline of improvement to reach the improvement level (e.g., how long it took for the above-mentioned training user's skin to improve by 0.3).

Products and/or actions may facilitate the improvement of the training users' skin conditions. In some embodiments, these products and/or actions taken by a training user may correspond to a skin condition of the training user. For example, a training user with a dry skin condition may apply moisturizing lotion daily to improve the skin condition. Products included in the training user skin condition improvement information 230 include nutritional supplements, vitamins, dietary supplements, neutraceutical supplements, topical creams and/or serums, beauty products, lotions (such as lotions with sun protection factor or SPF), effervescent tablets and/or powders, over the counter pharmaceuticals and/or medical devices, and diagnostic tools. Actions included in the training user skin condition improvement information 230 include physical activity, washing the face, steaming the face, applying the products mentioned above to the face, among others.

In some embodiments, the training set 200 further includes information about each training user, such as characteristics and environmental conditions. Characteristics about each training user may include measurements of and/or describe the training user's age, mental health, productivity, sleep, pollution, sexual and reproductive health, fertility, performance in sports, gastrointestinal microbiome, pain, cardiovascular health, pregnancy, post-natal health, immunity, disposition to and/or state of cancer, chronic inflammation, weight and/or obesity, eating disorders, substance use, access to healthcare, injury, vaccines, HIV and/or AIDS, nervous system, disposition to and/or history of stroke, lung disease, blood health (e.g., blood sugar, blood pressure), and non-communicable diseases (e.g., autoimmune disorders, heart disease, diabetes). The environmental conditions of the training user may be described by conditions of air quality (e.g., carbon dioxide concentrations, volatile organic compounds), temperature, ultraviolet radiation level, and humidity, among other parameters. The environmental conditions, in some embodiments, includes data obtained via the training user's client device, such as a location of the training user via a GPS and an itinerary of the training user via a calendar coupled to the training user's client device. For example, the skin condition recommendation engine 155 may determine conditions describing the environment around the training user based on training user's location via a GPS on the training user's client device. The characteristics and environmental conditions associated with a training user may be recorded at set intervals over a period of time (e.g., every day for five weeks, every few hours, once every week).

The training user face images 210, the training user skin conditions 220, the training user skin condition improvement information 230, and the training user information in the training set 200 may be considered a part of a positive training set or a negative training set. The positive training set includes products and/or actions that positively impact the skin condition of training users. For example, the training user skin condition improvement information 230 may indicate that a training user with oily skin washed the training user's face multiple times per day, improving the oily skin condition. Thus, the action of washing the face associated with the oily skin condition was positive for the training user. The negative training set includes products and/or actions that negatively impact and/or have no impact on training users. Continuing the above example, a different training user, perhaps of a different age, with oily skin may react negatively. Washing the different training user's oily face may result in breaking out in acne, or cause dry skin for example. In another example, washing the face may result in no change of a training user's oily skin, thus neither positively nor negatively affecting the training user. Accordingly, the training set 200 provides the machine learned model 170 with information about a training user, a set of images of the training user's face, one or more skin conditions associated with the training user's face, and products and/or actions taken to improve the associated skin conditions. The training set 200 may be categorized into a positive and a negative training set.

The skin condition recommendation engine 155 uses supervised or unsupervised machine learning to train the machine learned model 170 using the positive and/or negative training sets of the training set 200. Different machine learning techniques may be used in various embodiments, such as linear support vector machine (linear SVM), boosting for other algorithms (e.g., AdaBoost), neural networks, logistic regression, naïve Bayes, memory-based learning, random forests, bagged trees, decision trees, boosted trees, or boosted stumps. In one embodiment, the machine learned model 170 performs image processing operations on the training user face images 210 to identify one or more image features. Features include, for example, edges, corners (e.g., interest points), blobs (e.g., groups of interest points), and ridges within each of the images 210. The machine learned model 170 then correlates the training user skin conditions 220 reported to be associated with the training user face images 210 with the identified image features. Accordingly, the machine learned model 170 identifies features of the images of training user's faces corresponding to one or more skin conditions.

The machine learned model 170 also identifies relationships between the training user skin conditions 220 and the training user skin condition improvement information 230 to provide recommendations to users. In one embodiment, the machine learned model 170 generates a matrix, based on the training set 200, tracking each training user's improvement in skin condition. Each row of the matrix corresponds to a skin condition, characteristic (e.g., age), or environmental condition (e.g., high ultraviolet radiation) of the training user, and each column corresponds to a point in time. The machine learned model 170 creates improvement vectors, which represent each product and/or action in the training user skin condition improvement information 230, the vectors identifying the benefits that each product and/or action has demonstrated for each skin condition. Accordingly, the machine learned model 170 is trained to generate recommendations on products and/or actions for users to take, in response to identifying information about the user and skin conditions of the user.

The trained machine learned model 170, when applied to images of another user's (e.g., the user 110) face 240, identifies one or more skin conditions 250 of the user and outputs recommendations 260 that increase a likelihood of remedying the identified skin conditions. As described with respect to FIG. 1, the images of the user's face may be reported to the skin condition recommendation engine 155 via a client device (e.g., the client device 120) and/or automatically obtained by the client device from an external data source (e.g., a social network system). The user may be incentivized to provide images of the face to the skin condition recommendation engine 155 via gamification, rewards, and/or product offers. In some embodiments, the machine learned model 170 is applied to information about the user (e.g., characteristics and environmental conditions) and the set of images of the user's face to identify skin conditions and output recommendations for remedying the identified skin conditions.

The images of the user's face 240 include a plurality of images of the face of the user (e.g., the user 110), similar to the training user face images 210. The user may provide the skin condition recommendation engine 155 with at least one image of the face at regular intervals of time (e.g., once every morning for two weeks).

Based on the images of the user's face 240, the trained machine learned model 170 identifies one or more skin conditions of the user.

In some embodiments, the skin condition recommendation engine 155 may perform one or more pre-processing operations on the images of the user's face 240 prior to the machine learned model 170 identifying skin conditions of the user. For example, the images of the user's face 240 may be rotated, tinted, as well as adjusted for white balance, brightness, and contrast, among other operations. The various image processing operations may facilitate the skin condition recommendation engine 155's identifying of features in the images of the user's face 240. In some embodiments, the features may include edge detection features, texture features, skin color and/or tint, or some combination thereof, and may be associated with one or more skin conditions. The trained machine learned model 170 subsequently identifies the one or more skin conditions associated with the features identified within the images of the user's face 240.

In some embodiments, the machine learned model 170 identifies the skin conditions based on information about the user. For example, the machine learned model 170 may account for the age of the user when identifying the skin condition (e.g., people over the age of 60 are more likely to have dry skin). Accordingly, the trained machine learned model 170 identifies one or more skin conditions associated with the images of the user's face 240.

After identifying the one or more skin conditions of the user, the trained machine learned model 170 generates a matrix that describes the skin conditions and characteristics of the user. Similar to the matrix built from the training set 200, the matrix includes rows corresponding to a skin condition, characteristic, or environmental condition, and columns corresponding to points in time. The machine learned model 170 calculates a dot product of each improvement vector (e.g., a vector for each product and/or action that resulted in an improvement in skin conditions of the training users) and the user matrix. The resultant dot product with the highest value for each skin condition, characteristic, and/or environmental condition indicates which product and/or action has the highest likelihood of improving the user's skin conditions. Accordingly, the trained machine learned model 170 provides recommendations to the user for improving skin conditions associated with the user.

The recommendations 260 include suggested products and/or actions that will help the user improve the one or more skin conditions 250. Recommended products and/or actions may be similar to the products used and actions performed by training users, as included in the training user skin condition improvement information 230. For example, in response to receiving images of a user's face 240 and identifying a sensitive skin condition 250, the machine learned model 170 may provide recommendations 260 of moisturizing lotions formulated for people with sensitive skin.

In some embodiments, the machine learned model 170 outputs recommendations 260 based on information about the user. For example, the machine learned model 170 may determine that a location of the user (e.g., determined from a GPS of the user's client device) has high ultraviolet radiation and subsequently recommend a lotion with a high sun protection factor (SPF). In another example, the machine learned model 170 may account for the user's age, recommending different lotions for users over the age of 50 than those under the age of 50.

The recommendations 260 are displayed on the user's client device, in some embodiments, by modifying a display of the client device. In some embodiments, the client device notifies the user of the recommendations 260.

### Process for Presenting Recommendations

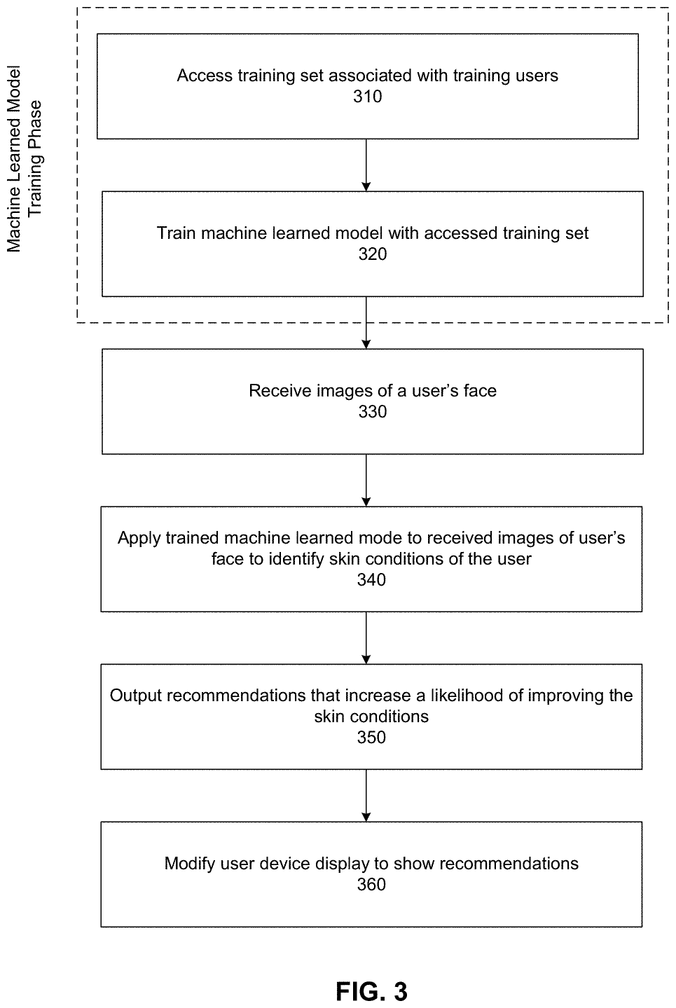

FIG. 3 illustrates an example process for providing a user with recommendations for improving skin conditions, in accordance with one or more embodiments. A skin condition recommendation engine (e.g., the skin condition recommendation engine 155) accesses 310 a training set (e.g., the training set 200) associated with a plurality of training users (e.g., the training users 140). For each training user, the training set includes images of the training user's face, one or more skin conditions of the training user, information about how the training user improved their skin conditions, and in some embodiments, information about the training user (e.g., demographic information, environmental conditions, etc.).

The skin condition recommendation engine trains 320 a machine learned model (e.g., the machine learned model 170) with the training set. The machine learned model determines features in the images of the training users' faces associated with one or more skin conditions. In some embodiments, the machine learned model also determines a relationship between products used and/or actions taken by the training users and improvements in their skin conditions.

The skin condition recommendation engine receives 330 images of a user's face (e.g., the images of the user's face 240), and in some embodiments, information about the user (e.g., demographic information, environmental conditions, etc.). The user (e.g., the user 110) is distinct from the training users. The images of the user's face and the information about the user may be captured and/or tracked over a period time.

The skin condition recommendation engine applies 340 the trained machine learned model to the received images of the user's face. The trained machine learned model identifies one or more skin conditions (e.g., the skin conditions 250) of the user, based on the received images of the user's face.

The skin condition recommendation engine outputs 350 recommendations that increase a likelihood of improving the user's skin conditions. The trained machine learned model identifies recommendations (e.g., the recommendations 260) based on the skin conditions of the user. The recommendations include products and/or actions that may help with improving the identified skin conditions. In some embodiments, the recommendations are based on the information about the user. The skin condition recommendation engine may also output, in some embodiments, lifestyle recommendations, in addition to skin condition related recommendations. Lifestyle recommendations may be based on the user and may include, for example, community service, environmental activities, and physical activity. For example, upon determining that the user's location is close to a beach, the skin condition recommendation engine may suggest activities such as a beach cleanup. In another embodiment, the skin condition recommendation engine also suggests, to the user, reducing the use of plastic. The skin condition recommendation engine may incentivize the user to follow through on recommended products and/or actions by gamification, rewards (such as social network status awards), and/or product offers.

The skin condition recommendation engine modifies 360 a display of a client device of the user (e.g., the client device 120) to include the recommendations that will aid the user in remedying the identified skin conditions.

In some embodiments, the user presents feedback to the skin condition recommendation engine as to whether the recommended products and/or actions helped with improving the skin conditions. The presented feedback is added to the training set to improve the machine learned model's recommendations, for example by retraining the machine learned model.

In some embodiments, the skin condition recommendation engine evaluates whether the recommendations are improving the identified skin conditions. The user provides the skin condition recommendation engine with images of the user's face regularly. The machine learned model is applied to the images of the user's face and outputs one or more skin conditions, represented by coefficients (e.g., percentages of the one or more skin conditions), associated with each image of the user's face. A change in the skin conditions and/or transition to normal skin may signify that the user's skin conditions improved. In some embodiments, the skin condition recommendation engine stitches together images of the user's face captured at regular intervals to form a video, animation, and/or GIF showing how the user's skin conditions have improved. In some embodiments, the skin condition recommendation engine recommends alternatives and/or new products and/or actions in response to determining that the skin conditions have not improved sufficiently.

### Example User Interface

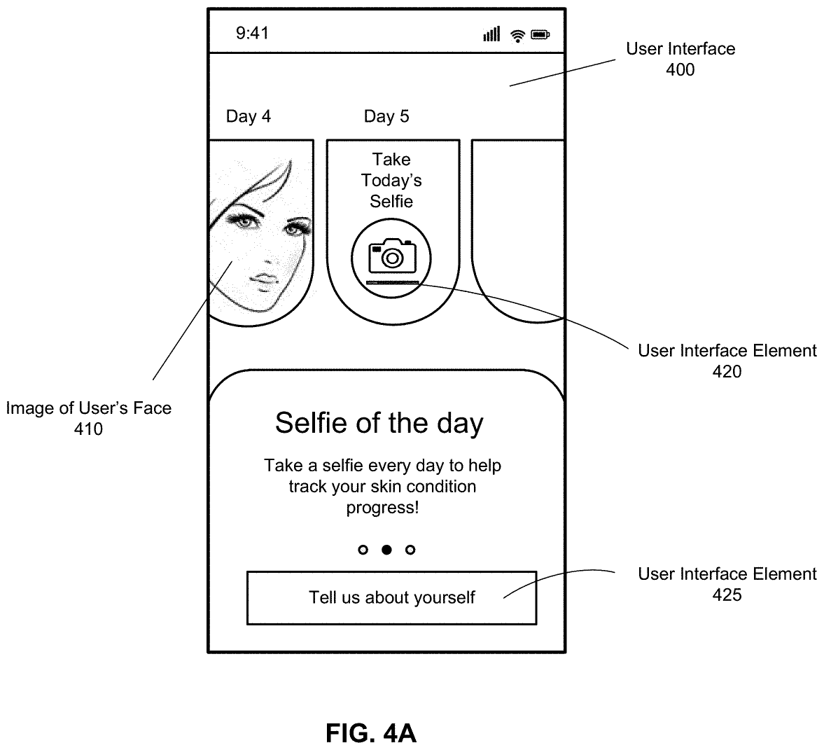

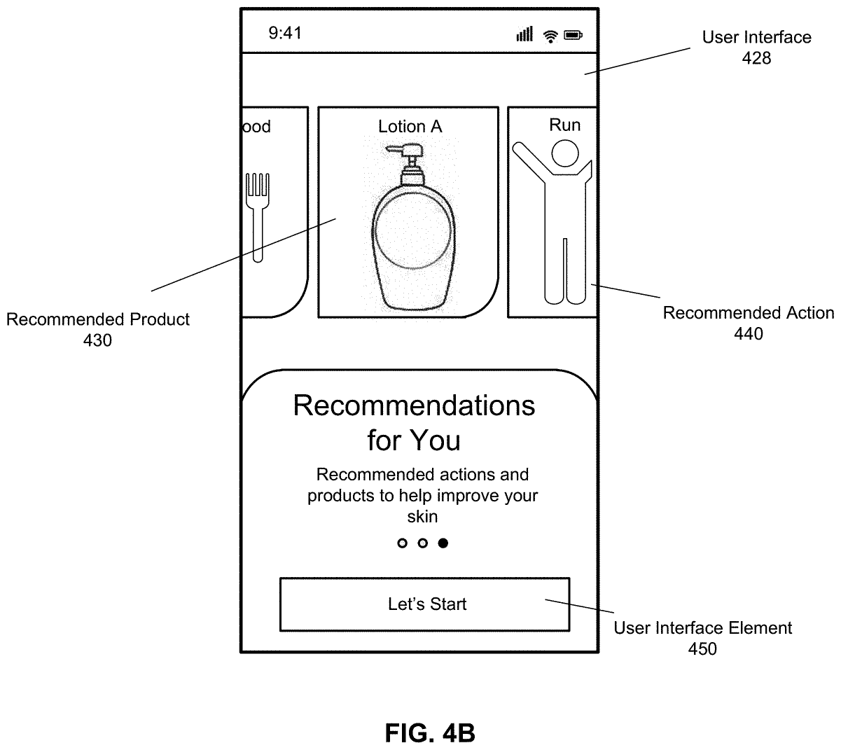

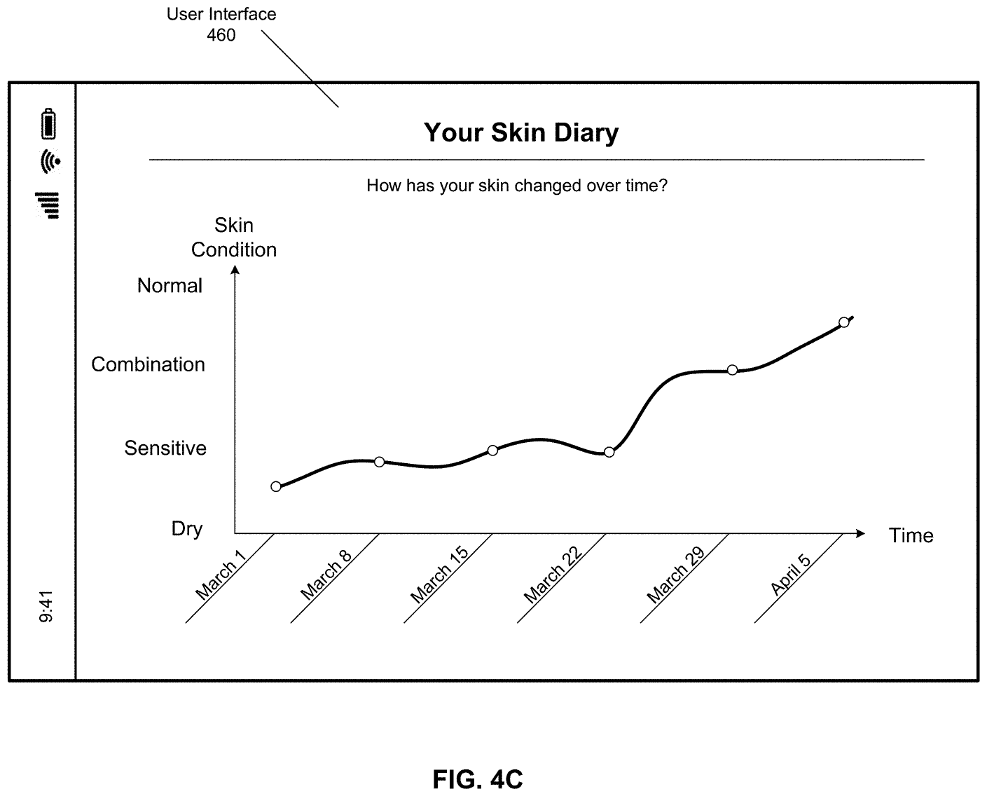

FIGS. 4A-C illustrate an example user interfaces through which a user may interact with the skin condition recommendation engine, in accordance with one or more embodiments. In some embodiments, the user accesses the skin condition recommendation engine via an application executed by a client device (e.g., the client device 120). The client device displays the user interface of the skin condition recommendation engine, and enables the user to provide input to and/or interact with the skin condition recommendation engine.

In FIG. 4A, the user interface 400 enables the user to provide one or more images of the user's face to the skin condition recommendation engine. As described with respect to FIG. 2, the skin condition recommendation engine applies a machine learned model to images of the user's face to identify one or more skin conditions of the user's face. In some embodiments, the machine learned model also accounts for information about the user when identifying skin conditions. The machine learned model accordingly recommends actions and/or products that increase a likelihood of the skin conditions being remedied.

The user interface 400 provides for the display of images of the user's face 410 captured over time at regular intervals (e.g., once every day for one month). In some embodiments, the client device captures the image of the user's face. In other embodiments, the user uploads the image of the user's face to the skin condition recommendation engine via the client device. When the user interacts with a user interface element 420, the user can capture a new image of the face. In some embodiments, with the user's consent, the skin condition recommendation engine adds the captured images of the user's face to a training set used to train the machine learned model.

The user interface 400 also includes a user interface element 425 that, when interacted with, enables the user to input information about the user. This information includes user characteristics (e.g., age, health conditions, dietary preferences, and so on) and environmental conditions (e.g., indicated by a location of the user). In some embodiments, the user provides permission, via the user interface element 425, for the skin condition recommendation engine to extract user information from another application hosted and/or executed on the client device (e.g., a fitness tracking application, a social networking application).

In FIG. 4B, the user interface 428 presents recommendations to the user to help alleviate skin conditions identified by the machine learned model. The recommendations include recommended products 430 (e.g., lotions, nutritional supplements, vitamins, and so on) and recommended actions 440 (e.g., exercise, ways to improve diet, face washing, and so on). The user interface 428 also includes user interface elements 450 that, when interacted with, provide further information on the recommended products 430 and the recommended actions 440. For example, in some embodiments, the user interface elements 450 allow the user to order one or more of the recommended products 430 via the skin condition recommendation engine. In some embodiments, the user interface 428 includes user interface elements that, when interacted with, share the recommendations to a social network of the user.

In FIG. 4C, the user interface 460 displays changes in skin conditions of the user tracked by the skin condition recommendation engine over time. In some embodiments, based on the images of the user's face input at regular intervals, the skin condition recommendation engine tracks improvements in the skin conditions of the user over time. In some embodiments, the skin condition recommendation engine further recommends products and/or actions based on the changes in the skin conditions over time. In some embodiments, the skin condition recommendation engine adds information on whether the recommended products 430 and/or recommended actions 440 were effective in improving the skin conditions of the user to the training set used to train the machine learned model.

### ADDITIONAL CONFIGURATION CONSIDERATIONS

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
accessing a training set of information comprising, for each of a plurality of training users, an image of the training user's face and an identification of one or more skin conditions of the training user;
training a machine-learned model based on the accessed training set of information, the machine-learned model configured to identify one or more skin conditions corresponding to a face based on images of the face;
receiving, from a user, a set of images of the user's face;
applying the machine-learned model to the received set of images of the user's face to identify one or more skin conditions of the user;
identifying one or more actions that, if performed by the user, increase a likelihood that the identified one or more skin conditions will be remedied; and
modifying an interface displayed by a device of the user to include a recommendation to the user to perform the one or more actions.

2. The method of claim 1, further comprising identifying one or more products that, if used by the user, increase a likelihood that the identified one or more skin conditions will be remedied, and wherein the recommendation further includes a recommendation to use the identified one or more products.

3. The method of claim 1, wherein the identified one or more skin conditions are selected from a set of skin conditions, wherein the machine-learned model is configured to assign a coefficient to each of the set of skin conditions based on an analysis of the received images, each coefficient corresponding to a likelihood of a presence of the skin condition, and wherein the identified one or more skin conditions comprise the skin conditions of the set of skin conditions assigned an above-threshold coefficient.

4. The method of claim 1, wherein the identified one or more skin conditions comprise one or more of: normal, sensitive, combination, oily, and dry.

5. The method of claim 1, wherein the one or more skin conditions of a training user are identified by a doctor.

6. The method of claim 1, wherein the one or more skin conditions of a training user are self-reported by the training user.

7. The method of claim 1, wherein the training set further comprises, for each of the plurality of training users, training user information describing characteristics of the training user and an environment of the training user.

8. The method of claim 7, further comprising:
receiving, from the user, user information describing characteristics of the user and an environment of the user;
applying the machine-learned model additionally to the received user information to identify the one or more actions.

9. The method of claim 1, wherein the identified one or more actions comprise the use of one or more products to increase a likelihood that the identified one or more skin conditions will be remedied.

10. The method of claim 1, wherein training the machine-learned model comprises, for each of the plurality of training users:
performing one or more image processing operations on the image of the training user's face;
identifying one or more image features of the processed images; and
correlating one or more skin conditions of the training user to the identified one or more image features.

11. The method of claim 10, wherein applying the machine-learned model comprises performing the one or more image processing operations on the received images of the user's face to identify image features of the received images.

12. The method of claim 1, further comprising:
receiving, from the user, a second set of images of the user's face;
determining, from the second set of images, a level of improvement of the identified one or more skin conditions;
generating, from the second set of images, an animation showing the level of improvement; and
modifying the interface to display the generated animation to the user.

13. The method of claim 1, wherein the set of images of the user's face is received in response to a request for the set of images by an application running on a client device of the user.

14. A non-transitory computer readable storage medium comprising computer executable code that when executed by one or more processors causes the one or more processors to perform operations comprising:
accessing a training set of information comprising, for each of a plurality of training users, an image of the training user's face and an identification of one or more skin conditions of the training user;
training a machine-learned model based on the accessed training set of information, the machine-learned model configured to identify one or more skin conditions corresponding to a face based on images of the face;
receiving, from a user, a set of images of the user's face;
applying the machine-learned model to the received set of images of the user's face to identify one or more skin conditions of the user;
identifying one or more actions that, if performed by the user, increase a likelihood that the identified one or more skin conditions will be remedied; and
modifying an interface displayed by a device of the user to include a recommendation to the user to perform the one or more actions.

15. The non-transitory computer readable storage medium of claim 14, the operations further comprising identifying one or more products that, if used by the user, increase a likelihood that the identified one or more skin conditions will be remedied, and wherein the recommendation further includes a recommendation to use the identified one or more products.

16. The non-transitory computer readable storage medium of claim 14, wherein the identified one or more skin conditions are selected from a set of skin conditions, wherein the machine-learned model is configured to assign a coefficient to each of the set of skin conditions based on an analysis of the received images, each coefficient corresponding to a likelihood of a presence of the skin condition, and wherein the identified one or more skin conditions comprise the skin conditions of the set of skin conditions assigned an above-threshold coefficient.

17. The non-transitory computer readable storage medium of claim 14, wherein the identified one or more skin conditions comprise one or more of: normal, sensitive, combination, oily, and dry.

18. The non-transitory computer readable storage medium of claim 14, wherein the one or more skin conditions of a training user are identified by a doctor.

19. The non-transitory computer readable storage medium of claim 14, wherein the one or more skin conditions of a training user are self-reported by the training user.

20. A computer system comprising:
one or more computer processors; and
a non-transitory computer readable storage medium comprising computer executable code that when executed by the one or more processors causes the one or more processors to perform operations comprising:
accessing a training set of information comprising, for each of a plurality of training users, an image of the training user's face and an identification of one or more skin conditions of the training user;
training a machine-learned model based on the accessed training set of information, the machine-learned model configured to identify one or more skin conditions corresponding to a face based on images of the face;
receiving, from a user, a set of images of the user's face;
applying the machine-learned model to the received set of images of the user's face to identify one or more skin conditions of the user;
identifying one or more actions that, if performed by the user, increase a likelihood that the identified one or more skin conditions will be remedied; and
modifying an interface displayed by a device of the user to include a recommendation to the user to perform the one or more actions.
